(19) United States
(12) Patent Application Publication
(10) Pub. No.: US 2010/0209375 A1
(43) Pub. Date: Aug. 19, 2010

(54) PROCESS FOR TREATING KERATIN FIBERS WITH AT LEAST ONE HETEROCYCLIC DISULFIDE ENTITY IN THE PRESENCE OF AT LEAST ONE REDUCING AGENT, OPTIONALLY HEAT, AND OPTIONALLY AT ALKALINE PH.

(76) Inventors: Maxime Deboni, Tokyo (JP); Hiroshi Takahashi, Tokyo (JP); Yosuke Hirasawa, Yokohama-shi (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/635,217

(22) Filed: Dec. 10, 2009

Related U.S. Application Data
(60) Provisional application No. 61/138,298, filed on Dec. 17, 2008.
(60) Provisional application No. 61/138,304, filed on Dec. 17, 2008.
(60) Provisional application No. 61/138,311, filed on Dec. 17, 2008.
(60) Provisional application No. 61/138,211, filed on Dec. 17, 2008.

(30) Foreign Application Priority Data
Dec. 11, 2008 (FR) 0858465
Dec. 11, 2008 (FR) 0858473
Dec. 11, 2008 (FR) 0858475

(51) Int. Cl. A61K 8/49; A61Q 5/04

(52) U.S. Cl. 424/70.5

(57) ABSTRACT

The present disclosure relates to a process for treating keratin fibers, comprising: applying to the keratin fibers at least one heterocyclic disulfide entity chosen from those of formula (I), optionally applying to the keratin fibers at least one reducing agent; and/or optionally heating the keratin fibers at a temperature ranging from 50 to 250° C.; optionally at an alkaline pH; it being understood that when the at least one heterocyclic disulfide entity chosen from those of formula (I) is lipoic acid, and at least one reducing agent is applied to the keratin fibers, then the lipoic acid is not present together in the same composition with the at least one reducing agent. The present disclosure also relates to a cosmetic composition, optionally at alkaline pH, comprising, in a cosmetic support: at least one heterocyclic disulfide entity chosen from those of formula (I), and optionally at least one reducing agent; it being understood that when the at least one heterocyclic disulfide entity chosen from those of formula (I) is lipoic acid, and at least one reducing agent is applied to the keratin fibers, then the lipoic acid is not present together in the same composition with the at least one reducing agent.

This application claims benefit of U.S. Provisional Application Nos. 61/138,298; 61/138,304; and 61/138,311, filed Dec. 17, 2008. This application also claims benefit of priority under 35 U.S.C. §119 to French Patent Application Nos. FR 0858465; FR 0858473; and FR 0858475, filed Dec. 11, 2008.

The present disclosure relates to the use, as an agent for treating keratin fibers and for example for permanently reshaping keratin fibers, i) of at least one heterocyclic disulfide entity as defined herein in the presence of ii) at least one reducing agent and/or iii) heat provided by heating at a temperature ranging from 50 to 250° C., and/or iv) at alkaline pH. The present disclosure also relates to a cosmetic composition comprising, in a cosmetic support, i) at least one heterocyclic disulfide entity as defined herein and/or i) at least one reducing agent and/or ii) at alkaline pH, and to a process for permanently reshaping keratin fibers and/or for reducing the degradation of keratin fibers, for example human keratin fibers such as the hair

A common technique for permanently reshaping the hair typically consists of, in a first stage, opening the —S—S— disulfide bonds of keratin (cystine) by applying to the hair, which has been placed under tension beforehand (with curlers or the like), a composition containing a reducing agent (reduction step) and then, after having rinsed the head of hair thus treated, reconstituting said disulfide bonds in a second stage by applying to the hair, which is still under tension, an oxidizing composition (oxidation step, also known as the fixing step) so as to finally give the hair the desired shape, known as the artificial shape. This technique thus makes it possible, without preference, to make the hair wavy, or to relax or uncurl it.

The holding of the artificial shape and the degradation of keratin fibers are two major problems in the development of products for transforming said fibers, for example by using reducing treatments in permanent-waving or straightening operations. Specifically, the two steps of reduction and oxidation performed in conventional permanent reshaping processes may, when they are repeated, give rise to degradation of the keratin fibers. Furthermore, the results in terms of artificial-shape hold obtained after performing these processes may be insufficient for the user. Another method for opening the disulfide bonds of keratin and also heterocyclic disulfide compounds for the permanent waving of hair is to use electromagnetic radiation (U.S. Pat. No. 5,858,179). This technique requires the preliminary use of an electromagnetic source to activate the heterocyclic disulfide compounds.

Moreover, certain methods are aimed at overcoming the problem of the odor of the permanent-waving operation by using disulfide compounds (EP 1 129 687). Another document mentions a process for permanent-waving the hair using a composition comprising lipoic acid and a reducing agent (WO 2008/110 223).

Current dyeing techniques often rely on oxidation dyeing. This dyeing process involves:

oxidation bases such as ortho- or para-phenylenediamines, ortho- or para-aminophenols and heterocyclic compounds, and

couplers such as aromatic meta-diamines, meta-aminophenols, meta-diphenols and certain heterocyclic compounds such as indole compounds.

Oxidation bases and couplers are known as “oxidation dyes.”

Oxidation bases and couplers are capable of reacting together in an oxidizing medium to generate colored polymerized species.

At the same time, the alkaline conditions of the oxidizing medium used in oxidation dyeing may make it possible:

to swell the keratin fiber scales so as to facilitate the penetration of the bases and couplers into the fiber, and

to dissolve the melanin, enabling the fiber to be lightened by 1 to 4 tones.

The latter phenomenon may impair the integrity of the fiber, for example during repeated treatment with these oxidizing alkaline compositions used alone or in combination with other chemical treatments such as alkaline relaxing, bleaching, or permanent waving.

It is known practice to use α-lipoic acid for example to reduce the oxidative damage of hair, to reduce the irritation of the scalp, and to soothe the sensitivity of the scalp (WO 2007/140 856). However, the results obtained with α-lipoic acid may be unsatisfactory, such as in terms of degradation of fibers treated with oxidizing agents. Moreover, a change in color, shade, and dyeing power may be noted when employing the treatment methods of the prior art.

There is thus a need for cosmetic compositions that may reduce the degradation of keratin fibers during the oxidative treatment of keratin fibers, such as dyeing, lightening, or bleaching, while at the same time avoid an appreciable change in the color of the fibers thus treated, for example as regards the shade and the power. There is also a need for cosmetic compositions that allow artificial shaping of keratin fibers which are easy to use, long-lasting, and may not have the drawbacks of accentuated degradation of keratin fibers on repeated application, poor hold over time, and lack of cosmeticity of the keratin fibers after treatment.

Accordingly, the present disclosure relates to achieving at least one of the aims discussed above. Thus, disclosed herein is a process for treating keratin fibers, such as the hair, comprising:

applying to the keratin fibers at least one cosmetic composition comprising, in a cosmetic support, at least one heterocyclic disulfide entity chosen from those of formula (I), salts thereof with an acid or a base, stereoisomers thereof, and solvates thereof,

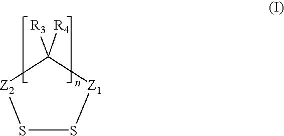

wherein

Z₁ and Z₂, which may be identical or different, represent a carbonyl group or a divalent group CR₁R₂ or CR₅R₆;

R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, xii) amino, xiii) aminocarbonyl(C₁-C₈)alkyl and xiv) (C₁-C₈)(alkyl)carbonyl(C₁-C₈)(alkyl)amino(C₁-C₈)alkyl; or alternatively

R₁ and R₃, or when n is 2 or 3 two radicals R₃ borne by adjacent carbon atoms, form, together with the carbon atoms that bear them, a cycloalkyl group, a heterocycloalkyl group, an aryl group such as phenyl, or a heteroaryl group;

n represents an integer equal to 1, 2 or 3, such as 1 or 2

it being understood that when n is 2 or 3, then the groups R₃ and R₄ may be identical or different;

optionally applying to the keratin fibers at least one reducing agent; and/or

optionally heating the keratin fibers at a temperature ranging from 50 to 250° C.;

wherein the at least one cosmetic composition comprising the at least one heterocyclic disulfide entity chosen from those of formula (I) optionally has an alkaline pH; and

further wherein the at least one reducing agent may be present in the at least one cosmetic composition comprising the at least one heterocyclic disulfide entity chosen from those of formula (I), or may be present in a separate cosmetic composition;

it being understood that when the at least one heterocyclic disulfide entity chosen from those of formula (I) is lipoic acid, and at least one reducing agent is applied to the keratin fibers, then the lipoic acid is not present together in the same composition with the at least one reducing agent.

The present disclosure also relates to a process for treating keratin fibers, comprising:

applying to the keratin fibers at least one cosmetic composition comprising, in a cosmetic support, at least one heterocyclic disulfide entity chosen from those of formula (I), salts thereof with an acid or a base, stereoisomers thereof, and solvates thereof;

optionally applying to the keratin fibers at least one reducing agent; and/or

optionally heating the keratin fibers at a temperature ranging from 50 to 250° C.;

wherein the at least one cosmetic composition comprising the at least one heterocyclic disulfide entity chosen from those of formula (I) optionally has an alkaline pH;

further wherein the at least one reducing agent may be present in the at least one cosmetic composition comprising the at least one heterocyclic disulfide entity chosen from those of formula (I), or may be present in a separate cosmetic composition;

it being understood that when the at least one heterocyclic disulfide entity chosen from those of formula (I) is lipoic acid, and at least one reducing agent is applied to the keratin fibers, then the lipoic acid is not present together in the same composition with the at least one reducing agent; and

further wherein the at least one heterocyclic disulfide entity chosen from those of formula (I) is chosen from those of formula (I′), salts thereof with an acid or a base, stereoisomers thereof, and solvates thereof,

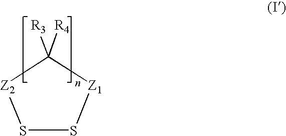

wherein:

Z₁ and Z₂, which may be identical or different, represent a carbonyl group or a divalent group CR₁R₂ or CR₅R₆;

R₁, R₂, R₅ and R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₃)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl,

R₃ and R₄, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl; or alternatively

R₁ and R₃, or when n is 2 or 3 two radicals R₃ borne by adjacent carbon atoms, form, together with the carbon atoms that bear them, a cycloalkyl group, a heterocycloalkyl group, an aryl group such as phenyl, or a heteroaryl group;

n represents an integer equal to 1, 2 or 3, such as 1 or 2;

it being understood that when n is 2 or 3, then the groups R₃ and R₄ may be identical or different; and

wherein the at least one cosmetic composition comprising the at least one heterocyclic disulfide entity chosen from those of formula (I′) further comprises at least one reducing agent.

The present disclosure also relates to a cosmetic composition, optionally at alkaline pH, comprising, in a cosmetic support, at least one heterocyclic disulfide entity chosen from those formulae (I) and (I′), salts thereof with an acid or a base, stereoisomers thereof, and solvates thereof, as defined herein; and optionally at least one reducing agent, it being understood that when the at least one heterocyclic disulfide entity chosen from those of formula (I) is lipoic acid, and at least one reducing agent is applied to the keratin fibers, then the lipoic acid is not present together in the same composition with the at least one reducing agent.

The present disclosure also relates to a cosmetic composition, optionally at alkaline pH, comprising, in a cosmetic support:

at least one reducing agent, and

at least one heterocyclic disulfide entity chosen from those of formula (I′), salts thereof with an acid or a base, stereoisomers thereof, and solvates thereof,

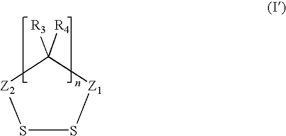

wherein:

Z₁ and Z₂, which may be identical or different, represent a carbonyl group or a divalent group CR₁R₂ or CR₅R₆;

R₁, R₂, R₅ and R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₃)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl;

R₃ and R₄, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl; or alternatively

R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;

n represents an integer equal to 1, 2 or 3;

it being understood that when n is 2 or 3, then the groups R₃ and R₄ may be identical or different.

The cosmetic composition according to the present disclosure may, for example, have an alkaline pH and further comprise at least one basifying agent. In at least one embodiment, the cosmetic composition may further comprise at least one oxidation base and optionally at least one coupler. In another embodiment, the cosmetic composition may further comprise at least one direct dye. In yet another embodiment, the cosmetic composition may further comprise at least one oxidizing agent.

The present disclosure also relates to a process for treating keratin fibers using:

i) at least one heterocyclic disulfide entity chosen from those of formulae (I) and (I′) as defined herein; and

ii) at least one reducing agent; and/or

iii) at alkaline pH; and/or

iv) in the presence of heat.

Such processes may for example be quick and simple to perform. In contrast to some processes known in the prior art, the at least one heterocyclic disulfide entity chosen from those of formulae (I) and (I′) as defined herein may not, for example, need to be subjected beforehand to the effect of an electromagnetic source in order to act in the permanent reshaping process.

The processes according to the present disclosure may be for example simple and quick. They also may have the advantage of transforming keratin fibers such as the hair aesthetically and durably with regard to the various external attacking factors, while at the same time maintaining very suitable fiber cosmeticity. Moreover, the processes according to the present disclosure may make it possible to obtain curling and wave hold that are more efficient than those of the standard methods not using disulfide compounds.

The use of at least one heterocyclic disulfide entity chosen from those of formulae (I) and (I′) as defined herein in alkaline medium may also make it possible to reduce the degradation of human keratin fibers such as hair, without appreciably changing the color of the fibers.

Other characteristics, aspects and benefits of the present disclosure will emerge more clearly on reading the detailed description that follows, and also the various concrete, but in no way limiting, examples intended to illustrate it.

Although the description that follows focuses for example on the case of treating hair, it will be noted here that the process according to the present disclosure can be applicable to any keratin fiber. The at least one heterocyclic disulfide entity chosen from those of formulae (I) and (I′) according to the present disclosure may be used for example on human keratin fibers such as hair.

### Heterocyclic Disulfide Compounds of Formulae (I) and (I′):

The at least one heterocyclic disulfide entity chosen from those of formulae (I) and (I′) as defined herein may be used according to the disclosure in the process for treating keratin fibers and/or may be contained in the cosmetic compositions disclosed herein.

The at least one heterocyclic disulfide entity chosen from those of formulae (I) and (I′) as defined herein may be in its “reduced” form (I)_{red} i.e. in its “open” form corresponding to its dithiol forms below:

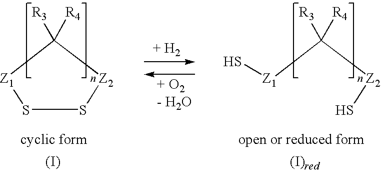

The reduced form (I)_{red} may be obtained from the in situ reduction of the at least one heterocyclic disulfide entity (I) in the presence of a reducing agent as defined herein, or alternatively the reduced form (I)_{red} may be used isolated in reduced form. This entity may then be in the at least one cosmetic composition in the presence of an oxidizing agent as defined herein, also to give in situ the cyclic form (I) or (I′).

For example, at least one of the radicals R₁ to R₆ of the at least one heterocyclic disulfide entity chosen from those of formulae (I) and (I′) may be other than a hydrogen atom.

According to at least one embodiment, the at least one heterocyclic disulfide entity chosen from those of formulae (I) and (I′) are such that R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) amino, and x) (C₁-C₈)(alkyl)carbonyl(C₁-C₈)(alkyl)amino(C₁-C₈)alkyl.

According to at least one embodiment, the at least one heterocyclic disulfide entity chosen from those of formula (I) and (I′) are such that n is 1 or 2. For example, the at least one heterocyclic disulfide entity chosen from those of formulae (I) and (I′) may be such that n is 1. According to another example, the at least one heterocyclic disulfide entity chosen from those of formulae (I) and (I′) may be such that n is 2.

For example, the at least one heterocyclic disulfide entity chosen from those of formula (I) may be such that R₁ to R₆ are chosen from hydrogen and hydroxy, carboxy and aminocarbonyl(C₁-C₃)alkyl groups.

For example, the at least one heterocyclic disulfide entity chosen from those of formula (I′) may be such that R₁ to R₆ are chosen from a hydrogen atom and hydroxyl and carboxy groups.

For example, the at least one heterocyclic disulfide entity chosen from those of formulae (I) and (I′) may be such that Z₁ represents CR₁R₂ and Z₂ represents CR₅R₆ with R₁, R₂, R₅ and R₆ chosen from a hydrogen atom and hydroxy, carboxy and aminocarbonyl(C₁-C₃)alkyl groups. For example, the at least one heterocyclic disulfide entity chosen from those of formulae (I) and (I′) may be such that R₃ and R₄, which may be identical or different, represent a hydrogen atom or a radical chosen from (C₁-C₃)alkyl, hydroxy and carboxy.

According to at least one embodiment, the at least one heterocyclic disulfide entity chosen from those of formulae (I) and (I′) may be such that Z₁ and/or Z₂ denote a carbonyl group, and/or R₁ and R₃ or both the radicals R₃ borne by adjacent carbon atoms form, together with the carbon atoms that bear them, a benzene ring.

It is understood that, in the definition of the radicals R₁ to R₆ of formulae (I) and (I′):

The alkyl radicals are chosen from saturated, linear and/or branched hydrocarbon-based radicals, such as of C₁-C₆ and for example of C₁-C₄, such as methyl, ethyl, propyl, butyl, pentyl and hexyl.

The alkoxy radicals are alkyl-oxy radicals with (C₁-C₆)alkyl for example of C₁-C₆ such as methoxy, ethoxy, propoxy, and butoxy.

The (poly)hydroxyalkyl radicals comprise on the (C₁-C₆)alkyl group as defined herein at least one substituted hydroxyl group; they may be for example hydroxy(C₁-C₆)alkyl radicals such as hydroxymethyl, hydroxyethyl and hydroxypropyl.

The cycloalkyl radicals are C₄-C₈ cycloalkyl radicals, which may contain an unsaturation, for example cyclopentyl and cyclohexyl radicals. The cycloalkyl radicals may also be substituted, for example, with alkyl, alkoxy, carboxylic acid, hydroxyl, amine, and ketone groups.

The heterocycloalkyl radicals are 4- to 8-membered radicals, containing from 1 to 3 identical or different heteroatoms, chosen from nitrogen, oxygen and sulfur and optionally containing from 1 to 3 carbon atoms. The heterocycloalkyl radicals may also be substituted, for example, with the same groups as those defined for the cycloalkyls.

The aryl radicals are monocyclic or polycyclic carbocyclic radicals, containing from 6 to 14 carbon atoms, and of which at least one ring is aromatic, such as phenyl, anthracenyl and xanthenyl, for example phenyl.

The heteroaryl radicals are 6- to 14-membered monocyclic or polycyclic cyclic radicals, containing from 1 to 3 identical or different heteroatoms, chosen from nitrogen, oxygen and sulfur and optionally containing from 1 to 3 carbon atoms. The heteroaryl radicals for example may be chosen from the following groups: pyridyl, indolyl, furyl.

The heteroaryl and aryl radicals may also be substituted, for example, with the same groups as those defined for the cycloalkyls.

The reduced derivatives are derivatives of the heterocyclic dithiol compounds in open forms of formula (I)_{red} as defined herein.

As non-limiting examples of the at least one heterocyclic disulfide entity chosen from those of formula (I), non-limiting mention may be made of compounds (I) to (6) listed below in Table I, stereoisomers thereof, salts thereof with an acid or a base, and solvates thereof such as hydrates:

TABLE I

| Name | CAS No. | Chemical formula |
|---|---|---|
| Lipoic acid | 62-46-4 | 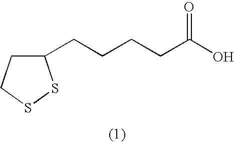 |
| Lipoamide | 940-69-2 | 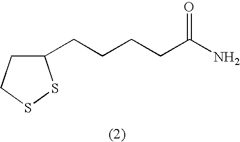 |
| Asparagusic acid | 2224-02-4 | 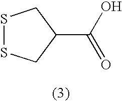 |
| 4,5-Dihydroxy- 1,2-dithiane | 14193-38-5 | 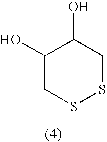 |
| 3H-1,2- Benzodithiol- 3-one | | 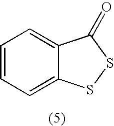 |
| 1,2-Dithiolane- 4-methyl-4- carboxylic acid | | 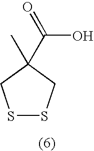 |

According to at least one embodiment, in its cyclic form, the at least one heterocyclic disulfide entity chosen from those of formulae (I) and (I′) has a logP value of less than 3.

The logP value represents the partition coefficient of the disulfide compound between octanol and water. It may be determined according to the method described in the article by Meylan and Howard “Atom/Fragment contribution method for estimating octanol-water partition coefficient,” J. Pharm. Sci., 84, 83-92 (1995). This value may also be calculated using commercially available software that determines the logP value from the structure of the molecule. An example that may be mentioned is the Epiwin software from the United States Environmental Agency.

The at least one heterocyclic disulfide entity chosen from those of formulae (I) and (I′) and the reduced forms thereof mentioned herein may be present in the at least one cosmetic composition of the disclosure in an amount ranging from 1% to 20% by weight, for example from 5% to 15% by weight, relative to the total weight of the composition.

As non-limiting examples of the at least one heterocyclic disulfide entity chosen from those of formula (I′) that may be used when the at least one cosmetic composition disclosed herein comprises at least one reducing agent and at least one heterocyclic entity chosen from those of formula (I′) as defined herein, mention may be made of compounds (3) to (6) listed in Table I, stereoisomers thereof, salts thereof with an acid or a base, and solvates thereof such as hydrates.

The acid included in the salts may be mineral or organic. As acids that may be used, non-limiting mention may be made of hydrochloric acid, sulfuric acid, phosphoric acid, acetic acid, tartaric acid, or methanesulfonic acid.

The base included in the salts may be mineral or organic. As bases that may be used, non-limiting mention may be made of sodium, potassium or ammonium hydroxide, sodium, potassium or ammonium carbonates or bicarbonates, basic amino acids such as arginine or lysine, and noncarboxylic organic amines and for example amino alcohols, for instance monoethanolamine or triethanolamine.

### Reducing Agents:

The process according to the disclosure may also include at least one reducing agent.

The term “reducing agent” means an agent that can reduce the disulfide functions of the keratin of keratin fibers. To do this, thiols for example may be used.

For instance, the thiols may be chosen from thioglycolic acid and salts thereof, glyceryl or glycol monothioglycolate, cysteamine and C₁-C₄ acyl derivatives thereof such as N-acetyl cysteamine or N-propionyl cysteamine, cysteine, N-acetylcysteine, cysteine esters, such as glyceryl cysteinate, N-mercaptoalkylamides of sugars such as N-(2-mercaptoethyl)gluconamide, thiolactic acid and its esters such as glyceryl monothiolactate, 3-mercaptopropionic acid and esters thereof, such as glyceryl 3-mercaptopropionate, thiomalic acid, 2-hydroxy-3-mercaptopropionic acid and esters thereof, such as glyceryl 2-hydroxy-3-mercaptopropionate, pantheteine, thioglycerol, the N-(mercaptoalkyl)-ω-hydroxyalkylamides described in patent application EP-A-354 835 and the N-mono- or N,N-dialkylmercapto-4-butryamides described in patent application EP-A-368 763, the aminomercaptoalkylamides described in patent application EP-A-432 000, the N-(mercaptoalkyl)succinamic acids or N-(mercaptoalkyl)succinimides described in patent application EP-A-465 342, the alkylaminomercaptoalkylamides described in patent application EP-A-514 282, the mixture of 2-hydroxypropyl thioglycolate and of 2-hydroxy-1-methylethyl thioglycolate described in patent application FR-A-2 679 448, and the N-mercaptoalkyl alkanediamides described in patent application EP-A-653 202.

Thioglycolic acid, thiolactic acid, cysteine and various derivatives thereof, cysteamine and derivatives thereof, 3-mercaptopropionic acid, and also esters thereof or salts thereof, for example glyceryl monothioglycolate, may also be used.

These active agents may be used alone or as a mixture.

The at least one reducing agent may generally be present in an amount ranging from 0.01% to 20% by weight relative to the weight of the cosmetic composition, for example ranging from 0.1% to 10% by weight relative to the weight of the composition.

### The Alkaline pH:

According to at least one embodiment, the cosmetic composition has an alkaline pH, i.e. it has a pH of greater than 7. For example, the pH may range from 8 to 14. According to at least one embodiment, the pH may range from 9 to 11.

To obtain this pH, at least one basifying agent as defined herein may be added to the cosmetic composition according to the disclosure comprising at least one heterocyclic disulfide entity chosen from those of formulae (I) and (I′) and the reduced forms thereof.

### Heat:

According to at least one embodiment, the process for treating keratin fibers may be performed in the presence of heat provided by heating the keratin fibers at a temperature ranging from 50° C. to 250° C., for instance from 50° C. to 100° C.

Heating throughout all or part of the process time may be performed using any device that produces heat and for example by devices such as a hairstyling hood, a hairdryer, or an infrared ray dispenser.

### The Process for Treating Keratin Fibers:

The treatment process according to the disclosure may be performed for the purpose of permanently reshaping keratin fibers such as the hair when it employs applying to the keratin fibers at least one heterocyclic disulfide entity chosen from those of formulae (I) and (I′) as defined herein and at least one reducing agent, and/or in the presence of heat. When the process employs applying to the keratin fibers the at least one heterocyclic disulfide entity chosen from those of formulae (I) and (I′) as defined herein at alkaline pH, it may then be performed for the purpose of reducing the degradation of the keratin fibers. It is understood that when the process employs applying the at least one heterocyclic disulfide entity chosen from those of formulae (I) and (I′) and at least one reducing agent and/or in the presence of heat and/or at alkaline pH, then permanent reshaping of keratin fibers may be performed while at the same time reducing the degradation of said fibers.

According to at least one embodiment, the process for permanently reshaping keratin fibers comprises applying to the keratin fibers to be treated:

at least one reducing agent; and

at least one heterocyclic disulfide entity chosen from those of formula (I), salts thereof with an acid or a base, stereoisomers thereof, and solvates thereof, as defined herein.

According to at least one embodiment of the disclosure, the process for permanently reshaping keratin fibers comprises:

applying at least one reducing agent to the keratin fibers to be treated,

rinsing the fibers, and

applying at least one heterocyclic disulfide entity chosen from those of formula (I) as defined herein to the fibers.

At least one embodiment of the disclosure comprises applying to keratin fibers a cosmetic composition comprising at least one reducing agent as defined herein and at least one heterocyclic disulfide entity chosen from those of formula (I′). For example, this process according to the present disclosure comprises:

applying to the keratin fibers to be treated a cosmetic composition comprising at least one reducing agent and at least one heterocyclic disulfide entity chosen from those of formula (I′) as defined herein; and

rinsing the fibers.

Devices and methods for placing the keratin fibers under mechanical tension may be used in the process. These devices and methods may be implemented before or during the application of the at least one reducing agent or before or during the application of the cosmetic composition comprising at least one reducing agent and at least one heterocyclic disulfide entity chosen from those of formula (I′). After applying the at least one heterocyclic disulfide entity chosen from those of formula (I) or the cosmetic composition comprising the at least one reducing agent and at least one heterocyclic disulfide entity chosen from those of formula (I′), the tensioning devices may be separated from the keratin fibers.

According to at least one embodiment of the process of the disclosure, the at least one reducing agent may be applied to wet hair. For example, the wet hair may be rolled up beforehand on tensioning devices such as rollers, for example 2 to 30 mm in diameter. The at least one reducing agent or the cosmetic composition comprising at least one reducing agent and at least one heterocyclic disulfide entity chosen from those of formula (I′) may also be applied gradually as the hair is rolled up, or alternatively, may be impregnated into the curler when it is made of foam, for example.

The at least one heterocyclic disulfide entity chosen from those of formula (I) may also be applied to the rolled-up hair, and can then react with the keratin fibers.

According to another embodiment, the cosmetic composition comprising the at least one reducing agent and the at least one heterocyclic disulfide entity chosen from those of formula (I′) may be applied to rolled-up hair.

Generally, the at least one reducing agent may be left on the fibers for a time period ranging from 5 to 60 minutes, for example from 15 to 45 minutes, and then rinsed thoroughly.

According to at least one embodiment of the disclosure, the application of the at least one heterocyclic disulfide entity chosen from those of formula (I) or of the cosmetic composition comprising at least one reducing agent and at least one heterocyclic disulfide entity chosen from those of formula (I′) may be followed by fixing the keratin fibers using at least one oxidizing agent.

Optionally, after applying the at least one heterocyclic disulfide entity chosen from those of formulae (I) and (I′) and/or applying the at least one oxidizing agent, the keratin fibers may be rinsed.

The at least one oxidizing agent may make it possible to reform the disulfide bonds of keratin fibers with the at least one heterocyclic disulfide entity chosen from those of formulae (I) and (I′) that have reacted. The leave-on time period may for example range from 2 to 10 minutes.

After applying the at least one heterocyclic disulfide entity chosen from those of formulae (I) and (I′), the keratin fibers may also be subjected to a heat treatment by heating at a temperature ranging from 50 to 250° C., such as 50° C. to 100° C., throughout all or part of the leave-on time. In practice, this operation may be performed using a hairstyling hood, a hairdryer, an infrared ray dispenser, or other standard heating appliances.

When it is desired to perform preliminary relaxing or curling of the hair, the process for treating keratin fibers may comprise applying at least one reducing agent to the hair, and then mechanically reshaping the hair via, for example, straightening the hair with a large-toothed comb, with the back of a comb, or with the hands. After a general leave-on time ranging from 5 to 60 minutes, for example from 15 to 45 minutes, straightening may be performed again, and the keratin fibers may then be rinsed thoroughly. At least one heterocyclic disulfide entity chosen from those of formulae (I) and (I′) then may be applied to the keratin fibers, which may or may not have been placed under tension. The keratin fibers may be optionally treated with at least one oxidizing agent that may be left on the fibers for a time period ranging from 2 to 10 minutes, and the fibers may then be rinsed thoroughly.

The process defined above may include drying the keratin fibers. The drying of the keratin fibers may be partial drying or full drying, and may be performed, for example, with a domestic hairdryer or alternatively a heating hood or bonnet, or else may be performed by thorough draining. For instance, the drying of the keratin fibers may be full drying. For example, a heating system may be used to dry the hair.

According to at least one embodiment of the present disclosure, the process for treating the keratin fibers according to the disclosure may be performed in the presence of heat and uses at least one heterocyclic disulfide entity chosen from those of formulae (I) and (I′) as defined herein. The presence of heat may be provided at any stage of the process and throughout the process.

The process of the present disclosure relates for example to the permanent reshaping of bleached keratin fibers such as bleached hair.

For example, the process for permanently reshaping keratin fibers according to the present disclosure may comprise

applying to the keratin fibers at least one heterocyclic disulfide entity chosen from those of formulae (I) and (I′), salts thereof with an acid or a base, stereoisomers thereof, and solvates thereof, as defined herein; and
heating the keratin fibers at a temperature ranging from 50° C. to 250° C., for example from 50° C. to 100° C., such as 80° C.

The heating time may range from 1 second to 4 hours, for example from 10 minutes to 3 hours, such as from 10 minutes to 2 hours.

According to at least one embodiment of the disclosure, the process comprises:

i) applying to the keratin fibers at least one heterocyclic disulfide entity chosen from those of formulae (I) and (I′) as defined herein,

ii) applying to the keratin fibers at least one reducing agent, and

iii) heating the keratin fibers at a temperature ranging from 50° C. to 250° C., for example at a temperature ranging from 50° C. to 100° C., such as 80° C.

According to at least one process of the disclosure, after applying the at least one heterocyclic disulfide entity chosen from those of formulae (I) and (I′), or the cosmetic composition comprising at least one reducing agent and at least one heterocyclic disulfide entity chosen from those of formulae (I) and (I′), the keratin fibers may be heated.

The keratin fibers may then be rinsed and dried.

According to at least one embodiment, the permanent reshaping process performed according to the present disclosure comprises:

applying at least one reducing agent as defined herein to the keratin fibers to be treated,

rinsing the keratin fibers,

applying to the keratin fibers at least one heterocyclic disulfide entity chosen from those of formulae (I) and (I′), salts thereof with an acid or a base, stereoisomers thereof, and solvates thereof, as defined herein, and

heating the keratin fibers.

According to another embodiment of the present disclosure, the process comprises:

applying a cosmetic composition comprising at least one reducing agent and at least one heterocyclic disulfide entity chosen from those of formulae (I) and (I′) as defined herein to the keratin fibers to be treated;

rinsing the keratin fibers, and

heating the keratin fibers.

Devices and methods for placing the keratin fibers under mechanical tension may be used in the process.

These devices and methods may be employed

a—before or during the application of the at least one heterocyclic disulfide entity chosen from those of formulae (I) and (I′); or

b—before or during the application of a cosmetic composition comprising at least one reducing agent and at least one heterocyclic disulfide entity chosen from those of formulae (I) and (I′); or

c—before heating.

The devices and methods for placing the keratin fibers under tension may then be separated from the keratin fibers. For instance, option a above may be chosen.

According to at least one embodiment of the process of the present disclosure, the hair may be moistened beforehand and rolled onto tensioning devices such as curlers. The curlers for example may have a diameter ranging from 2 to 30 mm. The at least one heterocyclic disulfide entity chosen from those of formulae (I) and (I′) or the cosmetic composition comprising at least one reducing agent and at least one heterocyclic disulfide entity chosen from those of formula (I′) may also be applied gradually as the hair is rolled up or alternatively may be impregnated into the curlers when they are made of foam, for example.

According to another embodiment, the at least one heterocyclic disulfide entity chosen from those of formula (I) or the cosmetic composition comprising at least one reducing agent and at least one heterocyclic disulfide entity chosen from those of formulae (I) and (I′) may be applied to the already rolled-up hair.

Generally, the at least one reducing agent may be left on the keratin fibers for a time period ranging from 5 to 60 minutes, for example from 15 to 45 minutes, and then rinsed thoroughly.

According to at least one embodiment of the process of the disclosure, the application of the at least one heterocyclic disulfide entity chosen from those of formulae (I) and (I′) may be followed by fixing the keratin fibers using at least one oxidizing agent.

Optionally, after applying the at least one heterocyclic disulfide entity chosen from those of formulae (I) and (I′) and/or applying the at least one oxidizing agent, the keratin fibers may be rinsed.

The at least one oxidizing agent may make it possible to reform the disulfide bonds of keratin with the heterocyclic disulfide entities chosen from those of formulae (I) and (I′) that have reacted. The leave-on time period may range from example from 2 to 10 minutes.

The keratin fibers may then be rinsed and dried.

When it is desired to perform preliminary relaxing or curling of the hair, at least one reducing agent is applied to the hair, and the hair may then be mechanically reshaped via a hair straightening operation with a large-toothed comb, with the back of a comb or with the hands. After a general leave-on time period ranging from 5 to 60 minutes, for example from 15 to 45 minutes, the hair may again be straightened and then rinsed thoroughly. At least one heterocyclic disulfide entity chosen from those of formulae (I) and (I′) is then applied to the keratin fibers, which have or have not been placed under tension. The at least one heterocyclic disulfide entity chosen from those of formulae (I) and (I′) may be left in place for a leave-on time period ranging from 1 second to 4 hours, for example from 10 minutes to 3 hours, such as from 10 minutes to 2 hours, under heat, and then the keratin fibers are optionally treated with at least one oxidizing agent, which may be left to act for a period of time ranging from 2 to 10 minutes, and the hair may then be rinsed thoroughly.

According to another embodiment of the process for treating keratin fibers in order to reduce the degradation of the fibers, the treatment process may employ at alkaline pH at least one heterocyclic disulfide entity chosen from those of formulae (I) and (I′) as defined herein and the reduced forms thereof. The at least one heterocyclic disulfide entity chosen from those of formulae (I) and (I′) may be in a cosmetic composition or may be used as a powder.

According to at least one embodiment of the present disclosure, the process for treating keratin fibers comprises:

applying to the keratin fibers a ready-to-use composition at alkaline pH comprising, in a cosmetic support, at least one heterocyclic disulfide entity chosen from those of formulae (I) and (I′) and the reduced forms thereof, salts thereof with an acid or a base, stereoisomers thereof, and solvates thereof, as defined herein, and at least one oxidizing agent; the ready-to-use composition having been prepared for example just before application by extemporaneous mixing of the at least one heterocyclic disulfide entity chosen from those of formulae (I) and (I′) and the reduced forms thereof (per se or present in a composition) and at least one oxidizing agent (per se or present in a composition);

leaving the ready-to-use composition on the keratin fibers for a period of time ranging from 1 minute to 2 hours; and

shampooing, rinsing and optionally drying the keratin fibers.

According to another embodiment of the present disclosure, the process for treating keratin fibers comprises:

applying to the keratin fibers at least one heterocyclic disulfide entity chosen from those of formulae (I) and (I′) and the reduced forms thereof, optionally in a ready-to-use cosmetic composition comprising it and as defined herein,

optionally leaving the at least one heterocyclic disulfide entity chosen from those of formulae (I) and (I′) on the keratin fibers for a period of time ranging from 1 minute to 2 hours,

applying to the keratin fibers at least one oxidizing agent, optionally in a cosmetic composition comprising it,

optionally leaving the at least one oxidizing agent for a period of time ranging from 1 minute to 2 hours, and

shampooing, rinsing and optionally drying the keratin fibers.

According to at least one embodiment, the process for treating keratin fibers according to the present disclosure also comprises applying to the keratin fibers at least one oxidation base as defined herein and optionally applying to the keratin fibers at least one coupler as defined herein, the at least one oxidation base and at least one coupler for example being present in the cosmetic composition comprising at least one heterocyclic disulfide entity chosen from those of formulae (I) and (I′) and the reduced forms thereof as defined herein.

According to at least one example of the process for treating keratin fibers of the present disclosure, before applying the at least one heterocyclic disulfide entity chosen from those of formulae (I) and (I′) and the reduced forms thereof, the keratin fibers are pretreated with at least one reducing agent as defined herein.

Generally, the at least one reducing agent may be left on the keratin fibers for a period of time ranging from 5 to 60 minutes, for example from 15 to 45 minutes, and then rinsed thoroughly.

The process defined herein may comprise drying the keratin fibers as defined herein.

The at least one reducing agent, the at least one heterocyclic disulfide entity chosen from those of formulae (I) and (I′) and the at least one oxidizing agent may be contained in separate cosmetic compositions or in a single cosmetic composition.

### The Compositions:

The cosmetic compositions according to the present disclosure comprise a cosmetic support generally containing water, a mixture of water and at least one organic solvent, or a mixture of organic solvents.

The term “organic solvent” means an organic substance that is capable of dissolving or dispersing in another substance without chemically modifying it.

### The Organic Solvents:

Non-limiting examples of organic solvents that may be mentioned include C₁-C₄ lower alcohols, such as ethanol and isopropanol; polyols and polyol ethers such as 2-butoxyethanol, propylene glycol, propylene glycol monomethyl ether, diethylene glycol monoethyl ether and monomethyl ether, hexylene glycol, and also aromatic alcohols, for instance benzyl alcohol or phenoxyethanol.

The organic solvents may be present in an amount for example ranging from 1% to 40% by weight, for instance from 5% to 30% by weight, relative to the total weight of the dye composition.

### Adjuvants:

The cosmetic composition(s) of the dyeing process in accordance with the present disclosure may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and for example anionic, cationic, nonionic and amphoteric polymeric associative thickeners, penetrants, sequestrants, fragrances, buffers, dispersants, conditioning agents, for instance volatile or nonvolatile, modified or unmodified silicones, film-forming agents, ceramides, preserving agents and opacifiers.

The adjuvants may be chosen for example from surfactants such as anionic or nonionic surfactants or mixtures thereof and mineral or organic thickeners.

The adjuvants may each be present in an amount ranging from 0.01% to 40% by weight relative to the weight of the composition, such as from 0.1% to 20% by weight relative to the weight of the composition.

Needless to say, a person skilled in the art will take care to select this or these additional compound(s) such that the beneficial properties intrinsically associated with the cosmetic composition(s) that is (are) useful in the reshaping process in accordance with the present disclosure are not, or are not substantially, adversely affected by the envisioned addition(s).

The cosmetic compositions used according to the disclosure, for example the one comprising at least one heterocyclic disulfide entity chosen from those formulae (I) and (I′), may also comprise at least one alkaline agent. These alkaline agents may for example contribute toward adjusting the pH.

### The Basifying Agents:

The at least one basifying agent that may be used in the cosmetic compositions according to the present disclosure is an agent for increasing the pH of the composition(s) in which it is present. The basifying agent may be a Bronsted, Lowry, or Lewis base. It may be mineral or organic.

For example, the basifying agent may be chosen from:

a) aqueous ammonia,

b) alkanolamines such as monoethanolamine, diethanolamine and triethanolamine, and also derivatives thereof,

c) oxyethylenated and/or oxypropylenated ethylenediamines,

d) mineral or organic hydroxides,

e) alkali metal silicates such as sodium metasilicates,

f) amino acids, such as basic amino acids, such as arginine, lysine, ornithine, citrulline and histidine,

g) (bi)carbonates such as of a primary, secondary or tertiary amine (ammonium) or of an alkali metal or alkaline-earth metal, and

h) the compounds of formula (II) below:

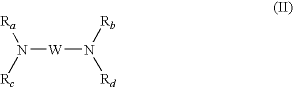

wherein W is a propylene residue optionally substituted with a hydroxyl group or a C₁-C₄ alkyl radical; and Rₐ, R_{b}, R_{c} and R_{d}, which may be identical or different, represent a hydrogen atom or a C₁-C₄ alkyl or C₁-C₄ hydroxyalkyl radical,

The mineral or organic hydroxides may for example be chosen from a) hydroxides of an alkali metal, b) hydroxides of an alkaline-earth metal, for instance sodium hydroxide or potassium hydroxide, c) hydroxides of a transition metal, such as hydroxides of metals from groups III, IV, V and VI, and d) hydroxides of lanthanides or actinides, quaternary ammonium hydroxides and guanidinium hydroxide.

The hydroxide may be formed in situ, for instance guanidine hydroxide, by reacting calcium hydroxide and guanidine carbonate.

The at least one basifying agent as defined herein may be present for example in an amount ranging from 0.001% to 10% by weight relative to the weight of the cosmetic composition, for example from 0.005% to 8% by weight of the composition.

When a cosmetic composition according to the present disclosure comprises at least one reducing agent, the pH of the cosmetic composition may range for example from 5 to 11.5, for example from 6 to 10.

The cosmetic compositions that may be used in the present disclosure may be in the form of a thickened or non-thickened lotion, a cream or gel or in any other suitable form and may contain additives that are known for their use in reducing compositions for permanently reshaping the hair.

The cosmetic compositions that may be used in the present disclosure may also be of the exothermic type, i.e. giving rise to a certain amount of heating during application to the hair, which gives a pleasant sensation to the person undergoing the permanent-waving operation. This cosmetic composition may also contain a solvent, for instance ethanol, propanol or isopropanol, glycerol, propylene glycol or diethylene glycol, at a maximum concentration of 20% relative to the total weight of the cosmetic composition.

Needless to say, a person skilled in the art will take care to select the optional additional compound(s) such that the beneficial properties intrinsically associated with the cosmetic composition(s) that is (are) useful in the reshaping process in accordance with the present disclosure are not, or are not substantially, adversely affected by the envisioned addition(s).

The pH of the cosmetic compositions may be conventionally adjusted by adding bases or at least one basifying agent as defined herein, or by adding acids.

The acids may be mineral or organic acids. They may for example be chosen from hydrochloric acid, orthophosphoric acid, sulfuric acid, carboxylic acids, for instance acetic acid, tartaric acid, citric acid or lactic acid, and sulfonic acids.

### The Oxidizing Agents:

In at least one embodiment of the present disclosure, the at least one heterocyclic disulfide entity chosen from those of formulae (I) and (I′) and the reduced forms thereof may be employed in the presence of at least one oxidizing agent, or alternatively the cosmetic composition according to the present disclosure at alkaline pH comprising at least one heterocyclic disulfide entity chosen from those of formulae (I) and (I′) may also contain at least one oxidizing agent (“ready-to-use” composition).

The at least one oxidizing agent may be chosen for example from aqueous hydrogen peroxide solution or hydrogen peroxide, alkali metal bromates, persalts such as persulfates, perborates or percarbonates, or polythionates, and mixtures of these oxidizing agents.

The hydrogen peroxide may be present in the cosmetic composition in an amount ranging from 0.1% to 20% by weight, for example from 0.1% to 10%; the alkali metal bromate may be present in the cosmetic composition in an amount ranging from 2% to 12% by weight; and the persalt may be present in the cosmetic composition in an amount ranging from 0.1% to 15% by weight, relative to the total weight of the cosmetic composition.

The at least one oxidizing agent may be in an oxidizing composition. The at least one oxidizing agent may be chosen for example from aqueous hydrogen peroxide solution or hydrogen peroxide, alkali metal bromates, persalts and polythionates, and a mixture of alkali metal bromate and persalt. The aqueous hydrogen peroxide solution may be present in the oxidizing composition in an amount ranging from 1 to 20 volumes, for example from 1 to 10 volumes; the alkali metal bromate may be present in the oxidizing composition in an amount ranging from 2% to 12% by weight; and the persalt may be present in the oxidizing composition in an amount ranging from 0.1% to 15% by weight, relative to the total weight of the oxidizing composition.

The pH of the oxidizing composition may for example range from 2 to 10, and is for example acidic, i.e. it ranges from 2 to 5.

### The Oxidation Dyes:

The process of the present disclosure may employ a dye composition comprising at least one oxidation base conventionally used in oxidation dyeing, or alternatively the cosmetic composition according to the present disclosure comprising at least one heterocyclic disulfide entity chosen from those of formula (I) may also contain at least one oxidation base conventionally used in oxidation dyeing.

By way of example, the at least one oxidation base may be chosen from para-phenylenediamines, bis(phenyl)alkylenediamines, para-aminophenols, bis-para-aminophenols, ortho-aminophenols, and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetyl-aminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, non-limiting mention may be made of para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)-tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diamino-phenoxy)-3,6-dioxaoctane, and the addition salts thereof with an acid.

Among the para-aminophenols that may be mentioned, for example, are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof with an acid.

Among the heterocyclic bases that may be mentioned, for example, are pyridine derivatives, pyrimidine derivatives, and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diamino-pyridine, and the addition salts thereof with an acid.

Other pyridine oxidation bases that are useful in the present disclosure are the 3-aminopyrazolo[1,5-a]pyridine oxidation bases or addition salts thereof described, for example, in patent application FR 2 801 308. Non-limiting examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-yl-pyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)-amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol and 3-aminopyrazolo[1,5-a]pyridin-7-ol, and the addition salts thereof with an acid or with a base.

Among the pyrimidine derivatives that may be mentioned are the compounds described, for example, in patents DE 2 359 399; JP 88-169 571; JP 05-63124; EP 0 770 375 or patent application WO 96/15765, for instance 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, pyrazolopyrimidine derivatives such as those mentioned in patent application FR-A-2 750 048, and among which non-limiting mention may be made of pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-amino-pyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)-ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,-N7,N7-tetramethylpyrazolo[1,5-a]pyrimidine-3,7-diamine and 3-amino-5-methyl-7-imidazolylpropylaminopyrazolo[1,5-a]pyrimidine, and the addition salts thereof with an acid, and the tautomeric forms thereof, when a tautomeric equilibrium exists.

Among the pyrazole derivatives that may be mentioned are, for example, the compounds described in patents DE 3 843 892 and DE 4 133 957, and patent applications WO 94/08969, WO 94/08970, FR-A-2 733 749 and DE 195 43 988, for instance 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

A 4,5-diaminopyrazole may for example be used, such as 4,5-diamino-1-(β-hydroxyethyl)pyrazole and/or a salt thereof.

Pyrazole derivatives that may also be mentioned include, for example, diamino-N,N-dihydropyrazolopyrazolones and for instance those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

2,3-Diamino-6,7-dihydro-1H,5H-pyrazolo[1,2-a]pyrazol-1-one and/or a salt thereof may for example be used.

Heterocyclic bases that may for example be used include 4,5-diamino-1-(n-hydroxyethyl)pyrazole and/or 2,3-diamino-6,7-dihydro-1H,5H-pyrazolo[1,2-a]pyrazol-1-one and/or a salt thereof.

The at least one oxidation base that may be used in the present disclosure and that may be present for instance in the cosmetic composition comprising at least one heterocyclic disulfide entity chosen from those of formula (I) and the reduced forms thereof may be each generally present in an amount ranging from 0.001% to 10% by weight, for example from 0.005% to 6% by weight relative to the total weight of the dye composition.

In general, the addition salts of the oxidation bases and couplers that may be used in the context of the present disclosure may for example be chosen from addition salts with an acid, such as hydrochlorides, hydrobromides, sulfates, citrates, succinates, tartrates, lactates, tosylates, benzenesulfonates, phosphates and acetates, and the addition salts with a base such as sodium hydroxide, potassium hydroxide, ammonia, amines or alkanolamines.

The present disclosure may employ at least one oxidation base as defined herein and also at least one coupler conventionally used in oxidation dyeing, or alternatively the cosmetic composition according to the present disclosure comprising at least one heterocyclic disulfide entity chosen from those of formulae (I) and (I′) and the reduced forms thereof, and at least one oxidation base, may also contain at least one coupler conventionally used for the dyeing of keratin fibers.

Among these couplers, non-limiting mention may be made for example of meta-phenylenediamines, meta-aminophenols, meta-diphenols, naphthalene-based couplers and heterocyclic couplers, and the addition salts thereof.

Non-limiting examples that may be mentioned include 2-methyl-5-aminophenol, 5-N-(β-hydroxyethyl)amino-2-methylphenol, 6-chloro-2-methyl-5-aminophenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine 3,5-diamino-2,6-dimethoxypyridine, 1-N-(β-hydroxyethyl)amino-3,4-methylenedioxybenzene and 2,6-bis(β-hydroxyethylamino)toluene, and the addition salts thereof with an acid.

In the cosmetic composition according to the present disclosure, the at least one coupler may be each generally present in an amount ranging from 0.001% to 10% by weight and for example from 0.005% to 6% by weight relative to the total weight of the dye composition.

### The Additional Direct Dyes:

At least one additional direct dye may also be present in the cosmetic composition according to the disclosure.

The at least one direct dye may be chosen, for example, from those conventionally used in direct dyeing, and among which non-limiting mention may be made of any commonly used aromatic and/or nonaromatic dye such as neutral, acidic or cationic nitrobenzene direct dyes, neutral, acidic or cationic azo direct dyes, neutral, acidic or cationic quinone and for example anthraquinone direct dyes, azine, triarylmethane, indoamine, methine, styryl, porphyrin, metalloporphyrin, phthalocyanine, cyanine and methine direct dyes, and fluorescent dyes.

Among the natural direct dyes, non-limiting mention may be made of lawsone, juglone, indigo, isatin, curcumin, spinulosin, apigenidin and orceins. It is also possible to use extracts or decoctions containing these natural dyes, and for example henna-based poultices or extracts.

In at least one example, the at least one direct dye may be chosen from Basic Red 51, Basic Orange 31, Basic Yellow 87, and Basic Red 22.

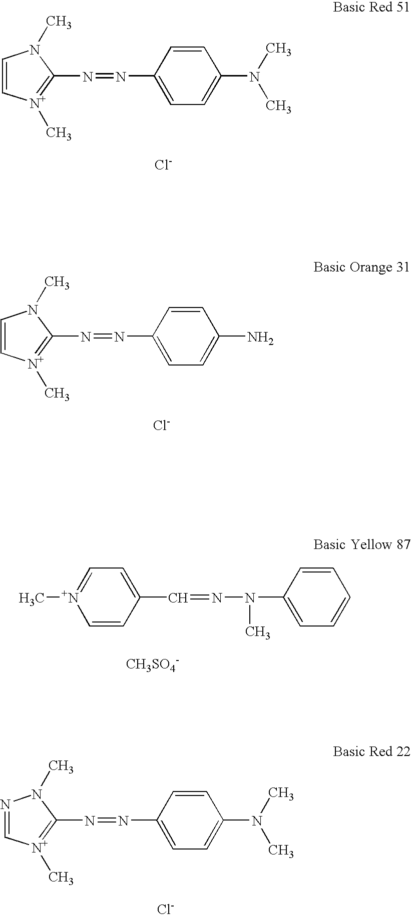

The other common characteristics of the cosmetic compositions of the present disclosure described herein apply to this oxidizing composition.

### The Tensioning Devices:

Placement of the keratin fibers under tension may be performed via any device or method, for example with elastic bands, clips, combs, hairgrips, or hair ties, or alternatively with conventional cylindrical curlers or rollers, and curlers of “tulip” type.

The curlers may be made of foam, for instance those described in document U.S. Pat. No. 5,992,425. In this case, the foam roller may be impregnated with a haircare product such as the cosmetic composition comprising at least one reducing agent or the cosmetic composition also comprising at least one heterocyclic disulfide entity chosen from those of formulae (I) and (I′) as defined herein, and on which the lock of keratin fibers is rolled up for the purpose of coating this lock with product over its entire rolling length. Curlers of “tulip” type are composed of an elongated stem forming a body terminated at one end with a head having at least one perforation. The body of the curler is made of a supple and flexible material, such that its free end can be introduced into the perforation of the head and be held therein by elastic gripping. A curler of the type defined above is described, for example, in patent application FR 2 602 650. The use of a “tulip” curler makes it possible to control the tension applied to the hair and thus to give a rounded shape to the locks of hair throughout the treatment.

In at least one embodiment, the tensioning device or method may be curlers ranging from 2 to 30 mm in diameter.

By way of non-limiting illustration, concrete examples of certain embodiments of the present disclosure are given below.

### EXAMPLES OF PERMANENT RESHAPING

### Examples 1 and 2

### Compositions

The following compositions were prepared (amounts expressed in grams of active material):

Composition 1 (Reducing Lotion):

| Ingredients | g (AM) |
|---|---|
| Thioglycolic acid | 7 |
| Aqueous ammonia (aqueous 20% ammonia solution) | 9.1 |
| Ammonium bicarbonate | 2.5 |
| Ammonium chloride | 1.7 |
| Diethylenetriaminepentaacetic acid (sodium salt | 0.4 |
| at 40% in water) | |
| Demineralized water | qs 100 |

Compositions 2 and 3 (Intra-Permanent) Comprising at Least One Heterocyclic Disulfide Entity Chosen from Those of Formula (I):

| | Composition 2 | Composition 3 |
|---|---|---|
| Ingredients | g (AM) | g (AM) |
| Lipoic acid | 0.5 | — |
| Trans-4,5-dihydroxy-1,2-dithiane | — | 10 |
| Monoethanolamine | qs pH 8 | qs pH 6.5 |
| Demineralized water | qs 100 | qs 100 |

Composition 4 (Fixing/Oxidizing Composition) Comprising at Least One Oxidizing Agent:

| Ingredients | g (AM) |
|---|---|
| Hydrogen peroxide at 35% in water | 5.15 |
| Sodium salicylate | 0.01 |
| 8-Hydroxyquinoline sulfate | 0.0125 |
| Citric acid | qs pH 3 |
| Demineralized water | qs 100 |

Application Process

A 1 g lock of hair was rolled up on a curler 17 mm in diameter. The reducing composition 1 was then applied to the lock in a bath ratio of 1.5 (1.5 grams of formulation per 1 gram of hair) and left on for a period of 15 minutes. The lock was rinsed with clear water. An intra-permanent composition (composition 2 or 3) was then applied to the lock in a bath ratio of 1.5 and left on for a period of 10 minutes. Next, oxidizing composition 4 was applied to the lock in a bath ratio of 1.5 and left on for a period of 5 minutes. Finally, the lock of hair was unwound, rinsed with clear water, and then dried.

The above application process without the application of an intra-permanent composition was used as the reference test.

### Example 1

A lock of natural Caucasian hair (tone height of 3) and a lock of bleached hair (bleached for 30 minutes at 35° C. using a bleaching composition formed from a weight for weight mixture of Platifiz Precision and 20-volumes aqueous hydrogen peroxide solution, which are commercial references from L'Oréal Professionnel) were permanent-waved according to the process described above, using composition 2 as the intra-permanent composition.

The locks thus treated were compared with reference locks (treated without applying an intra-permanent composition) in terms of curling and curl-hold performance.

The cosmetic composition and the process of the disclosure afforded a better curl result than the standard process, whether the hair was natural or bleached.

A curl hold test was performed by keeping the locks under tension for 5 hours at 40° C. and at 100% relative humidity. After the hold test, the locks treated according to the above permanent-waving process of the present disclosure had curl yields higher than the reference locks.

### Example 2

A lock of natural Caucasian hair (tone height of 3) and a lock of bleached hair (bleached as described in Example 1) were permanent-waved according to the process indicated above, this time using composition 3 as the intra-permanent composition.

The locks thus treated were compared with the reference locks (treated without applying an intra-permanent composition) in terms of curling and curl-hold performance.

The cosmetic composition and the process of the present disclosure offered a better curl result than the standard process, whether on natural or bleached hair.

The locks thus permanent-waved were subjected to the curl hold test described in Example 1. The curling results obtained with the permanent-waving treatment of the disclosure were more long-lasting than the curling results obtained according to the reference treatment.

### Example 3

### Comparative with Reducing Lotion Containing Lipoic Acid

The following reducing composition was prepared (contents expressed in grams of active material):

Composition 5 (Comparative Reducing Containing Lipoic Acid):

| Ingredients | g (AM) |
|---|---|
| Thioglycolic acid | 7 |
| Aqueous ammonia (aqueous 20% ammonia solution) | 9.1 |
| Lipoic acid | 0.5 |
| Ammonium bicarbonate | 2.5 |
| Ammonium chloride | 1.7 |
| Diethylenetriaminepentaacetic acid (sodium salt at 40% in water) | 0.4 |
| Demineralized water | qs 100 |

Application Process A, According to the Present Disclosure

A 1 g lock of Caucasian natural hair (tone height of 3) was rolled up on a curler 17 mm in diameter. The reducing composition 1 was applied in a bath ratio of 1.5 (1.5 grams of formulation per 1 gram of hair) and left on for a period of 15 minutes. The lock was rinsed with clear water. Intra-permanent composition 2 was then applied in a bath ratio of 1.5 and left on for a period of 10 minutes. Next, oxidizing composition 4 was applied in a bath ratio of 1.5 and left on for a period of 5 minutes. Finally, the lock of hair was unwound, rinsed with clear water, and then dried.

Results for the Process A According to the Present Disclosure:

The cosmetic composition and the process according to the present disclosure offered a very good homogeneity and curl result. Moreover, the hair was very smooth to the touch.

Application Process B, Using Composition 5:

A 1 g lock of Caucasian natural hair (tone height of 3) was rolled up on a curler 17 mm in diameter. The reducing composition 5 was applied in a bath ratio of 1.5 (1.5 grams of formulation per 1 gram of hair) and left on for a period of 15 minutes. The lock was rinsed with clear water. Intra-permanent composition 2 was then applied in a bath ratio of 1.5 and left on for a period of 10 minutes. Next, oxidizing composition 4 was applied in a bath ratio of 1.5 and left on for a period of 5 minutes. Finally, the lock of hair was unwound, rinsed with clear water, and then dried.

Results for the Process B According to the Comparative:

In terms of curl quality, the comparative process B offered lower cosmetic results than the process according to the disclosure, for example in terms of the responsiveness and homogeneity of the curl. The feel of the lock treated with comparative process B remained unchanged without any increase in the feel (for example, in terms of smoothness), unlike the lock treated with inventive process A.

### Example 4

### Composition with Lipoic Acid and Lipoamid in a Reducing Medium

Compositions 6 and 7 (Reducing Lotions):

| | Composition 6 | Composition 7 |
|---|---|---|
| Ingredients | g (AM) | g (AM) |
| Thioglycolic acid | 7 | 7 |
| Lipoic acid | 0.1 | — |
| Lipoamide | — | 0.1 |
| Aqueous ammonia (aqueous 20% | 9.1 | 9.1 |
| ammonia solution) | | |
| Ammonium bicarbonate | 2.5 | 2.5 |
| Ammonium chloride | 1.7 | 1.7 |
| Diethylenetriaminepentaacetic acid | 0.4 | 0.4 |
| (sodium salt at 40% in water) | | |
| Demineralized water | qs 100 | qs 100 |

Application Process

A lock of natural Japanese hair was permanent-waved according to the following process:

One g locks of hair were rolled up on curlers 17 mm in diameter. Reducing compositions 6 and 7 were applied to separate locks in a bath ratio of 1.5 (1.5 grams of formulation per 1 gram of hair) and left on for a period of 15 minutes. After the leave-on time, the locks were rinsed with clear water. Next, oxidizing composition 4 was applied in a bath ratio of 1.5 and left on for a period of 5 minutes. Finally, the locks of hair were rinsed with clear water, unwound, and then dried.

The curl obtained using reducing composition 7 (with lipoamide) was more homogeneous and possessed a better aesthetic quality than the one obtained with reducing composition 6 (with lipoic acid).

### Examples 5, 6 and 7

### Compositions

The following compositions were prepared (contents expressed in grams of active material):

| | Compo- | Compo- | Compo- | |
|---|---|---|---|---|
| | sition | sition | sition | Composition |
| Ingredients | 3′ | 4′ | 5′ | 6′ |
| Thioglycolic acid | 1 | — | — | — |
| Diammonium | — | 2.1 | — | — |
| dithioglycolate (as an | | | | |
| aqueous 48% solution | | | | |
| Lipoic acid | — | — | 10 | — |
| Trans-4,5-dihydroxy- | — | — | — | 3 |
| 1,2-dithiane | | | | |
| Aqueous ammonia | qs pH 8.8 | qs pH 8.8 | qs pH 8.8 | qs pH 10 |
| (aqueous solution | | | | |
| containing 20% | | | | |
| ammonia) | | | | |
| Demineralized water | qs 100 | qs 100 | qs 100 | qs 100 |

### Example 5

On 1 g locks of natural Japanese hair and bleached Japanese hair (bleached for 30 minutes at 35° C. using a bleaching composition formed from a weight for weight mixture of Platifiz Precision and of 20-volumes aqueous hydrogen peroxide solution, which are commercial references from L'Oréal Professionnel) rolled up on 17 mm curlers, composition 3′ was applied in a bath ratio of 2 and left on for a period of 2 hours at 80° C. (machine from Digital Perm, Oohiro ODIS-2). After the leave-on time, the locks were rinsed and then dried.

The locks thus treated were compared with locks treated with compositions 4′ and 5′ under the same conditions.

Composition 5′ allowed the hair to be shaped aesthetically. The shapings obtained with composition 3′ were heterogeneous. Those obtained with composition 4′ were very poor.

### Example 6

On 1 g locks of natural Japanese hair and bleached Japanese hair (bleached according to the bleaching treatment of Example 5) rolled up on 17 mm curlers, composition 6′ was applied in a bath ratio of 2 and left on for a period of two hours at 80° C. (machine from Digital Perm, Oohiro ODIS-2). After the leave-on time, the locks were rinsed and then dried.

The locks thus treated were compared with locks treated with composition 3′ under the same conditions.

Composition 6′ was more efficient than composition 3′ in terms of curling performance. The shapings obtained with composition 3′ were heterogeneous.

The locks treated with compositions 3′ and 6′ were immersed for 4 minutes in an aqueous solution at 40° C. containing 8% sodium lauryl ether sulfate. The curling results obtained with composition 6′ were longer lasting and more aesthetic than those obtained with composition 3′.

### Example 7

On 1 g locks of natural Japanese hair and bleached Japanese hair (bleached according to the bleaching treatment of Example 5 above) rolled up on 1.7 mm curlers, composition 5′ containing 10% lipoic acid was applied in a bath ratio of 2 and left on for a period of 2 hours at 80° C. After the leave-on time, each lock was rinsed and then dried.

The locks thus treated were compared with locks treated with the same composition 5′ and left on for a period of 2 hours at room temperature.

The locks treated with composition 5′ in the presence of heat had aesthetic, homogeneous, long-lasting curls. The locks of hair treated with composition 5′ in the absence of heat, i.e. at room temperature (25° C.), showed no change in their shape.

### Examples 8 and 9

### Example 8

The following compositions were prepared (in mass %) as follows:

| | Inventive | Comparative | Control |
|---|---|---|---|
| | Composition | Composition | Composition |
| Ingredients | 7′ | 8′ | 9′ |
| Propylene glycol | 10 | 10 | 10 |
| Hydroxyethylcellulose | 1.5 | 1.5 | 1.5 |
| (MW 720 000) | | | |
| 1,2-Dithiolane-4-methyl-4- | 3.2 | — | — |
| carboxylic acid | | | |
| Lipoic acid | — | 4.0 | — |
| Aqueous ammonia (aqueous | qs | qs | qs |
| solution containing 25% | pH 10 | pH 10 | pH 10 |
| ammonia) | | | |
| Distilled water | qs 100 | qs 100 | qs 100 |

| | Oxidizing |
|---|---|
| Ingredients | composition |
| Hydrogen peroxide | 6 |
| Pentasodium pentetate | 0.5 |
| Distilled water | qs 100 |

Composition 7′ corresponds to a cosmetic composition according to the present disclosure. Composition 8′ is a comparative composition containing lipoic acid. Composition 9′, which corresponds to the control, does not contain any heterocyclic disulfide entity.

Each of the compositions 7′ to 9′ was mixed weight for weight with the oxidizing composition, and then applied to locks of natural Caucasian hair containing 90% gray hairs, in a bath ratio of 2, and left on for a period of 30 minutes at room temperature.

After the leave-on time, the hair was shampooed, rinsed, and then dried at 40° C. The same operation was performed five times in total.

The cysteic acid (fiber integrity marker) at the surface of the hair was measured by Fourier-Transform Infrared analysis (Thermo Nicolet AVATAR 330 FT-IR). The analysis conditions are described in the following manner:

Scanning=64 scans

Resolution: 4 cm⁻¹

Wavenumbers measured: 800-2000 cm⁻¹

Mode: absorbance

Number of measurements per treatment: 15

The amount of cysteic acid is estimated by the ratio of the height at the wavenumber of the amide function (1654 cm⁻¹) to the height at the wavenumber of the cysteic acid function (1043 cm⁻¹).

The smaller the amount of cysteic acid, the less degradation there is at the surface of the fibers.

The following surface cysteic acid values were obtained:

| Composition | Surface cysteic acid |
|---|---|
| 7′ (inventive) | 0.036 |
| 8′ (comparative) | 0.050 |
| 9′ (control) | 0.059 |

From the above table, it is seen that the application of compositions 8′ and 9′ of the comparative test and of the control, respectively, to the hair gave rise to significantly more degradation of the fibers than when the same fibers were treated with composition 7′ according to the disclosure.

### Example 9

The following compositions were prepared (in mass %):

| | | | Compar- | |
|---|---|---|---|---|
| | Inventive | Inventive | ative | Control |
| | Compo- | Compo- | Compo- | Compo- |
| | sition | sition | sition | sition |
| Ingredients | 10′ | 11′ | 12′ | 13′ |
| Propylene glycol | 10 | 10 | 10 | 10 |
| Hydroxyethylcellulose | 1.5 | 1.5 | 1.5 | 1.5 |
| (MW 720 000) | | | | |
| 4,5-Dihydroxy-1,2- | 3.0 | | — | — |
| dithiane | | | | |
| 3H-1,2-Benzodithian- | — | 3.3 | | — |
| 3-one | | | | |
| Lipoic acid | — | — | 4.0 | — |
| Aqueous ammonia | qs | qs | qs | qs |
| (aqueous solution | pH 10 | pH 10 | pH 10 | pH 10 |
| containing 26.6% | | | | |
| ammonia) | | | | |
| Distilled water | qs 100 | qs 100 | qs 100 | qs 100 |

| Ingredients | Oxidizing composition |
|---|---|
| Hydrogen peroxide | 6 |
| Pentasodium pentetate | 0.5 |
| Distilled water | qs 100 |

Each of the compositions 10′ to 13′ was mixed weight for weight with the oxidizing composition, and then applied to locks of natural Caucasian hair containing 90% gray hairs, in a bath ratio of 2, and left on for a period of 30 minutes at room temperature.

After the leave-on time, the hair was shampooed, rinsed, and then dried at 40° C. The same operation was performed five times in total.

The diametric swelling of the hair in water was measured for each of the treated locks (Mitutoyo VL-50A micrometer).

In this method, the more the diameter of the hair increases, the greater the water uptake, which is evidence of impairment of the hair. The swelling rate is also calculated.

Maximum swelling and swelling rate values are provided in the following table:

| | Maximum | |
|---|---|---|
| | diametric | Swelling rate |
| | swelling (%) | (%/min) |
| Natural hair | 9.1 ± 1.6 | 5.3 ± 2.4 |
| Composition 10′ (inventive) | 11.0 ± 1.6 | 24.3 ± 3.0 |
| Composition 11′ (inventive) | 10.4 ± 1.8 | 23.6 ± 5.8 |
| Composition 12′ (comparative) | 13.1 ± 2.5 | 28.8 ± 6.3 |
| Composition 13′ (control) | 12.6 ± 2.3 | 27.9 ± 7.6 |

Whether as regards the “maximum swelling” parameter or the “swelling rate” parameter, the values obtained for the keratin fibers treated using the compositions according to the present disclosure (compositions 10′ and 11′) indicate significant protection of the fiber during the treatment process, when compared with the fibers treated with the comparative composition, i.e. with the composition comprising lipoic acid, and the control composition not comprising any heterocyclic disulfide entity (compositions 12′ and 13′, respectively).

What is claimed is:

1. A process for treating keratin fibers, comprising:
applying to the keratin fibers at least one cosmetic composition comprising, in a cosmetic support, at least one heterocyclic disulfide entity chosen from those of formula (I), the reduced forms thereof, salts thereof with an acid or a base, stereoisomers thereof, and solvates thereof, 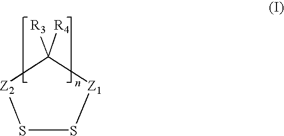
wherein:
Z₁ and Z₂, which may be identical or different, represent a carbonyl group or a divalent group CR₁R₂ or CR₅R₆;
R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, xii) amino, xiii) aminocarbonyl(C₁-C₈)alkyl and xiv) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different;
optionally applying to the keratin fibers at least one reducing agent; and/or
optionally heating the keratin fibers at a temperature ranging from 50 to 250° C.;
wherein the at least one cosmetic composition comprising the at least one heterocyclic disulfide entity chosen from those of formula (I) optionally has an alkaline pH; and
further wherein the at least one reducing agent may be present in the at least one cosmetic composition comprising the at least one heterocyclic disulfide entity chosen from those of formula (I), or may be present in a separate cosmetic composition;
it being understood that when the at least one heterocyclic disulfide entity chosen from those of formula (I) is lipoic acid, and at least one reducing agent is applied to the keratin fibers, then the lipoic acid is not present together in the same composition with the at least one reducing agent.

2. The process for treating keratin fibers according to claim 1, wherein the at least one heterocyclic disulfide entity chosen from those of formula (I) is chosen from those of formula (I′), salts thereof with an acid or a base, stereoisomers thereof, and solvates thereof, 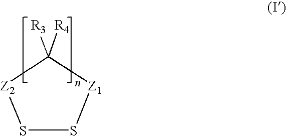
wherein:
Z₁ and Z₂, which may be identical or different, represent a carbonyl group or a divalent group CR₁R₂ or CR₅R₆;
R₁, R₂, R₅ and R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₃)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl;
R₃ and R₄, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different; and
wherein the at least one cosmetic composition comprising the at least one heterocyclic disulfide entity chosen from those of formula (I′) further comprises at least one reducing agent.

3. The process for treating keratin fibers according to claim 1, wherein the at least one heterocyclic disulfide entity chosen from those of formula (I) is such that R₁ to R₃, or when n is 2 or 3 two radicals R₃ borne by adjacent carbon atoms, form, together with the carbon atoms that bear them, an aryl group chosen from phenyl.

4. The process for treating keratin fibers according to claim 1, wherein the at least one heterocyclic disulfide entity chosen from those formula (I) is such that n is 1.

5. The process for treating keratin fibers according to claim 1, wherein the at least one heterocyclic disulfide entity chosen from those of formula (I) is such that n is 2.

6. The process for treating keratin fibers according to claim 1, wherein the at least one heterocyclic disulfide entity chosen from those of formula (I) is such that R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) amino, and x) (C₁-C₈)(alkyl)carbonyl(C₁-C₈)(alkyl)amino(C₁-C₈)alkyl.

7. The process for treating keratin fibers according to claim 1, wherein the at least one heterocyclic disulfide entity chosen from those of formula (I) is such that Z₁ represents CR₁R₂ and Z₂ represents CR₅R₆ with R₁, R₂, R₅ and R₆ chosen from a hydrogen atom and hydroxy, carboxy, and aminocarbonyl(C₁-C₃)alkyl groups.

8. The process for treating keratin fibers according to claim 1, wherein the at least one heterocyclic disulfide entity chosen from those of formula (I) is such that R₃ and R₄, which may be identical or different, represent a hydrogen atom or a radical chosen from (C₁-C₃)alkyl, hydroxyl, and carboxy.

9. The process for treating keratin fibers according to claim 1, wherein the at least one heterocyclic entity chosen from those of formula (I) is such that Z₁ and/or Z₂ denote(s) a carbonyl group, and/or R₁ and R₃ or two radicals R₃ borne by adjacent carbon atoms form, together with the carbon atoms that bear them, a benzene ring.

10. The process for treating keratin fibers according to claim 1, wherein the at least one heterocyclic disulfide entity chosen from those of formula (I) is chosen from lipoic acid, lipoamide, asparagusic acid, 4,5-dihydroxy-1,2-dithiane, 3H-1,2-benzodithiol-3-one, and 1,2-dithiolane-4-methyl-4-carboxylic acid, stereoisomers thereof, salts thereof with an acid or a base, and solvates thereof, wherein:

| Name | Chemical formula |
|---|---|
| Lipoic acid | 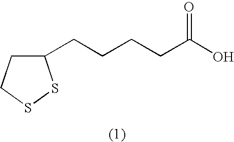 |
| Lipoamide | 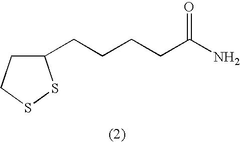 |
| Asparagusic acid | 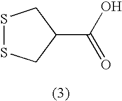 |
| 4,5-Dihydroxy-1,2-dithiane | 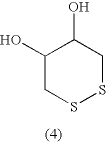 |
| 3H-1,2-Benzodithiol-3-one | 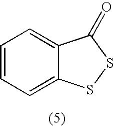 |
| 1,2-Dithiolane-4-methyl-4- carboxylic acid | 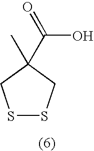 |

11. The process for treating keratin fibers according to claim 1, wherein the at least one heterocyclic disulfide entity chosen from those of formula (I), in its cyclic form, has a logP value of less than 3.

12. The process for treating keratin fibers according to claim 1, wherein the at least one heterocyclic disulfide entity chosen from those of formula (I) is present in the at least one cosmetic composition in an amount ranging from 1% to 20% by weight relative to the total weight of the composition.

13. The process for treating keratin fibers according to claim 1, wherein the at least one cosmetic composition further comprises at least one basifying agent chosen from:
a) aqueous ammonia,
b) alkanolamines,
c) oxyethylenated and/or oxypropylenated ethylenediamines,
d) mineral or organic hydroxides,
e) alkali metal silicates,
f) amino acids,
g) (bi)carbonates, and
h) the compounds of formula (II): 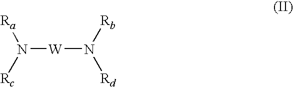
wherein:
W is a propylene residue optionally substituted with a hydroxyl group or a C₁-C₄ alkyl radical; Rₐ, R_{b}, R_{c} and R_{d}, which may be identical or different, represent a hydrogen atom or a C₁-C₄ alkyl or C₁-C₄ hydroxyalkyl radical.

14. The process for treating keratin fibers according to claim 13, wherein the at least one basifying agent is chosen from alkanolamines chosen from monoethanolamine, diethanolamine, and triethanolamine, and derivatives thereof.

15. The process for treating keratin fibers according to claim 13, wherein the at least one basifying agent is chosen from basic amino acids.

16. The process for treating keratin fibers according to claim 13, wherein the at least one basifying agent is chosen from (bi)carbonates of a primary, secondary, and tertiary amines (ammonium); of an alkali metals; and alkaline-earth metals.

17. The process for treating keratin fibers according to claim 1, wherein the at least one reducing agent is chosen from thioglycolic acid and salts thereof, glyceryl or glycol monothioglycolate, cysteamine and C₁-C₄ acyl derivatives thereof, cysteine, N-acetylcysteine, cysteine esters, N-mercaptoalkylamides of sugars, thiolactic acid and its esters, 3-mercaptopropionic acid and esters thereof, thiomalic acid, 2-hydroxy-3-mercaptopropionic acid and esters thereof, pantheteine, thioglycerol, N-(mercaptoalkyl)-w-hydroxyalkylamides and the N-mono- or N,N-dialkylmercapto-4-butryamides, aminomercaptoalkylamides, N-(mercaptoalkyl)succinamic acids or N-(mercapto-alkyl)succinimides, alkylaminomercaptoalkylamides, the mixture of 2-hydroxypropyl thioglycolate and of 2-hydroxy-1-methylethyl thioglycolate, and N-mercaptoalkyl alkanediamides.

18. The process for treating keratin fibers according to claim 1, wherein the at least one cosmetic composition comprises at least one reducing agent in an amount ranging from 0.01% to 20% by weight relative to the weight of the composition.

19. The process for treating keratin fibers according to claim 1, wherein the at least one cosmetic composition has an alkaline pH.

20. A cosmetic composition, optionally at alkaline pH, comprising, in a cosmetic support:
at least one heterocyclic disulfide entity chosen from those of formula (I) and the reduced forms thereof, salts thereof with an acid or a base, stereoisomers thereof, and solvates thereof, 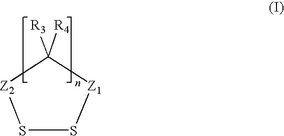
wherein:
Z₁ and Z₂, which may be identical or different, represent a carbonyl group or a divalent group CR₁R₂ or CR₅R₆;
R₁ to R₆, which may be identical or different, represent a hydrogen atom or a group chosen from: i) (C₁-C₈)alkyl, ii) aryl, hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, xii) amino, xiii) aminocarbonyl(C₁-C₈)alkyl and xiv) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different; and
optionally at least one reducing agent;
it being understood that when the at least one heterocyclic disulfide entity chosen from those of formula (I) is lipoic acid, then the lipoic acid is not present together in the same composition with the at least one reducing agent.

21. The cosmetic composition according to claim 20, further comprising at least one reducing agent, and wherein the at least one heterocyclic disulfide entity chosen from those of formula (I) is chosen from those of formula (I′) and the reduced forms thereof, salts thereof with an acid or a base, stereoisomers thereof, and solvates thereof, 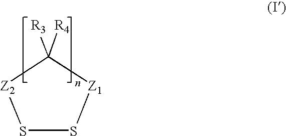
wherein:
Z₁ and Z₂, which may be identical or different, represent a carbonyl group or a divalent group CR₁R₂ or CR₅R₆;
R₁, R₂, R₅ and R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₃)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl;
R₃ and R₄, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, and xii) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different.

22. The cosmetic composition according to claim 20, wherein the cosmetic composition is at alkaline pH and further comprises at least one basifying agent.

23. The cosmetic composition according to claim 20, further comprising at least one oxidation base and optionally at least one coupler.

24. The cosmetic composition according to claim 20, further comprising at least one direct dye.

25. The cosmetic composition according to claim 20, further comprising at least one oxidizing agent.

26. A process for treating keratin fibers, comprising applying to human keratin fibers a ready-to-use cosmetic composition at alkaline pH comprising, in a cosmetic support:
at least one heterocyclic disulfide entity chosen from those of formula (I) and the reduced forms thereof, salts thereof with an acid or a base, stereoisomers thereof, and solvates thereof, 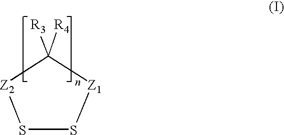
wherein:
Z₁ and Z₂, which may be identical or different, represent a carbonyl group or a divalent group CR₁R₂ or CR₅R₆;
R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, xii) amino, xiii) aminocarbonyl(C₁-C₈)alkyl and xiv) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different; and
at least one oxidizing agent.

27. The process for treating keratin fibers according to claim 26, further comprising:
leaving the ready-to-use composition on the keratin fibers for a period of time ranging from 1 minute to 2 hours; and
shampooing, rinsing, and optionally drying the keratin fibers.

28. A process for permanently reshaping keratin fibers, comprising applying to the keratin fibers to be treated:
at least one reducing agent; and
at least one heterocyclic disulfide entity chosen from those of formula (I), salts thereof with an acid or a base, stereoisomers thereof, and solvates thereof, 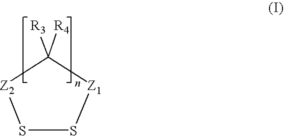
wherein:
Z₁ and Z₂, which may be identical or different, represent a carbonyl group or a divalent group CR₁R₂ or CR₅R₆;
R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, xii) amino, xiii) aminocarbonyl(C₁-C₈)alkyl and xiv) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different; and
it being understood that when the at least one heterocyclic disulfide entity chosen from those of formula (I) is lipoic acid, and at least one reducing agent is applied to the keratin fibers, then the lipoic acid is not applied together with the at least one reducing agent.

29. The process for permanently reshaping keratin fibers according to claim 28, further comprising:
rinsing the keratin fibers after applying the at least one reducing agent and before applying the at least one heterocyclic disulfide entity chosen from those of formula (I).

30. The process for treating keratin fibers according to claim 28, further comprising fixing the keratin fibers by applying to the keratin fibers at least one oxidizing agent, and optionally rinsing the fibers.

31. The process for treating keratin fibers according to claim 28, further comprising heating at a temperature ranging from 50° C. to 250° C.

32. The process for treating keratin fibers according to claim 31, wherein the heating temperature ranges from 50° C. to 100° C.

33. A process for permanently reshaping keratin fibers, comprising:
applying to the keratin fibers at least one heterocyclic disulfide entity chosen from those of formula (I), salts thereof with an acid or a base, stereoisomers thereof, and solvates thereof, 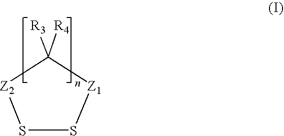
wherein:
Z₁ and Z₂, which may be identical or different, represent a carbonyl group or a divalent group CR₁R₂ or CR₅R₆;
R₁ to R₆, which may be identical or different, represent a hydrogen atom or a group chosen from: i) (C₁-C₈)alkyl, ii) aryl, iii) hydroxy, iv) (di)(C₁-C₈)(alkyl)amino, v) (C₁-C₈)alkoxy, vi) (poly)hydroxy(C₁-C₈)alkyl, vii) (di)(C₁-C₈)(alkyl)amino(C₁-C₈)alkyl, viii) carboxy, ix) carboxy(C₁-C₈)alkyl, x) (di)(C₁-C₈)(alkyl)aminocarbonyl(C₁-C₈)alkyl, xii) amino, xiii) aminocarbonyl(C₁-C₈)alkyl and xiv) (C₁-C₈)(alkyl)carbonyl(C₁-C₈)(alkyl)amino(C₁-C₈)alkyl; or alternatively
R₁ and R₃, or when n is 2 or 3 two radicals R₃ borne by adjacent carbon atoms, form, together with the carbon atoms that bear them, a cycloalkyl group, a heterocycloalkyl group, an aryl group, or a heteroaryl group;
n represents an integer equal to 1, 2 or 3;
it being understood that when n is 2 or 3, then the groups R₃ and R₄ may be identical or different; and
heating the keratin fibers at a temperature ranging from 50 to 250° C.

34. The process for treating keratin fibers according to claim 33, wherein the heating temperature ranges from 50° C. to 100° C.
